(19) United States
(12) Patent Application Publication
(10) Pub. No.: US 2024/0189600 A1
(43) Pub. Date: Jun. 13, 2024

(54) METHOD AND APPARATUS FOR VERY-HIGH FREQUENCY NEUROSTIMULATION

(71) Applicant: Boston Scientific Neuromodulation Corporation, Valencia, CA (US)

(72) Inventors: Rafael Carbunaru, Valley Village, CA (US); Hemant Bokil, Cambridge, MA (US); G. Karl Steinke, Valencia, CA (US)

(21) Appl. No.: 18/535,088

(22) Filed: Dec. 11, 2023

Related U.S. Application Data
(60) Provisional application No. 63/431,972, filed on Dec. 12, 2022.

(51) Int. Cl. A61N 1/36; A61N 1/06

(52) U.S. Cl. CPC A61N 1/36142; A61N 1/06; A61N 1/36125

(57) ABSTRACT

An example a neurostimulation system may include one or more sensors configured to sense a measure of tissue heating caused by neurostimulation, a stimulation output circuit configured to deliver the neurostimulation, and a stimulation control circuit configured to control the delivery of the neurostimulation using stimulation parameters. The stimulation control circuit may include temperature sensing circuitry and stimulation parameter circuitry. The temperature sensing circuitry may be configured to receive the measure of tissue heating and to determine a temperature parameter representing a temperature or a temperature change using the received measure of tissue heating. The stimulation parameter circuitry may be configured to adjust the stimulation parameters using the temperature parameter and to adjust the stimulation parameters to limit a dosage (energy or charge) based on an operational capability of the stimulation output circuit and the power management circuit and a safety limit related to tissue heating.

### CLAIM OF PRIORITY

This application claims the benefit of U.S. Provisional Application No. 63/431,972, filed on Dec. 12, 2022, which is hereby incorporated by reference in its entirety.

### TECHNICAL FIELD

This document relates generally to neurostimulation and more particularly to a system for delivering neurostimulation with very-high-frequency stimuli to a patient.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

In one example, the neurostimulation energy is delivered in a form of electrical pulses. The delivery is controlled using stimulation parameters that specify spatial (where to stimulate), temporal (when to stimulate), and informational (patterns of pulses directing the nervous system to respond as desired) aspects of the electrical pulses. These stimulation parameters are determined for therapeutic efficacy while ensuring patient safety. A neurostimulation system (e.g., including the implantable neurostimulator and the external programming device) needs to be capable of delivering the electrical pulses according to the stimulation parameters and capable of determining and programming the stimulation parameters to control the delivery.

### SUMMARY

An example (e.g., “Example 1”) of a system for delivering neurostimulation to one or more regions of tissue in a patient is provided. The system may include one or more sensors, a stimulation output circuit, and a stimulation control circuit. The one or more sensors may be configured to be placed in the patient to sense a measure of tissue heating caused by the delivery of the neurostimulation. The stimulation output circuit may be configured to deliver the neurostimulation. The stimulation control circuit may be configured to control the delivery of the neurostimulation using stimulation parameters and may include temperature sensing circuitry and stimulation parameter circuitry. The temperature sensing circuitry may be configured to receive the measure of tissue heating and to determine a temperature parameter representing a temperature or a temperature change using the received measure of tissue heating. The stimulation parameter circuitry may be configured to adjust the stimulation parameters using the temperature parameter and to adjust the stimulation parameters to limit a dosage based on an operational capability of the stimulation output circuit and the power management circuit and a safety limit related to tissue heating. The dosage may include an amount of at least one of an electrical energy or electrical charge injected in to the tissue by the delivery of the neurostimulation over a specified duration.

In Example 2, the subject matter of Example 1 may optionally be configured such that the stimulation output circuit is configured to deliver the neurostimulation using a very-high-frequency (VHF) stimulation waveform to the patient. The VHF stimulation waveform has a frequency of at least 100 kHz.

In Example 3, the subject matter of Example 2 may optionally be configured to include an implantable neurostimulator including at least the stimulation output circuit, the power management circuit, and the stimulation control circuit.

In Example 4, the subject matter of Example 3 may optionally be configured to include an external programmer configured to be communicatively coupled to the implantable neurostimulator. The external programmer includes a stimulation programming circuit configured to determine a pattern of neurostimulation pulses defined by the stimulation parameters. The stimulation control circuit is configured to control the delivery of the neurostimulation according to the pattern of neurostimulation pulses.

In Example 5, the subject matter of any one or any combination of Examples 3 and 4 may optionally be configured to further include one or more heat dissipators, a thermal management device configured to be coupled to the one or more heat dissipators, and a thermal conductive lead having a proximal end configured to be coupled to the thermal management device and a distal end configured to be placed in or about a region of the one or more regions of tissue. The thermal management device is configured to provide a thermal conductive path for a portion of a thermal energy causing the tissue heating to be dissipated through at least the one or more heat dissipators.

In Example 6, the subject matter of Example 5 may optionally be configured such that the implantable neurostimulator further includes the thermal management device.

In Example 7, the subject matter of Example 6 may optionally be configured such that the one or more heat dissipators include at least one implantable heat dissipator configured to be coupled to the implantable neurostimulator.

In Example 8, the subject matter of any one or any combination of Examples 6 and 7 may optionally be configured such that the implantable neurostimulator has a case housing at least the stimulation output circuit, the power management circuit, the stimulation control circuit, and the thermal management device, and may optionally be configured such that the one or more heat dissipators includes at least the case.

In Example 9, the subject matter of any one or any combination of Examples 5 to 8 may optionally be configured to further include one or more implantable leads each including a proximal end configured to be coupled to the implantable neurostimulator, a distal end configured to be placed in or about a region of the one or more regions of tissue, and a plurality of electrodes at the distal end. At least one of the one or more implantable leads is configured to be the thermal conductive lead. The one or more sensors are each incorporated into the distal end of a lead of the one or more implantable leads. The stimulation output circuit is configured to deliver the neurostimulation to the one or more regions of tissue using one or more electrodes selected from the plurality of electrodes at the distal end of each lead of the one or more implantable leads.

In Example 10, the subject matter of any one or any combination of Examples 5 to 9 may optionally be configured to further include a coolant circulation path formed in the one or more heat dissipators, the thermal management device, and the thermal conductive lead. The thermal management device includes a coolant reservoir coupled to the coolant circulation path and configured to store a coolant and a coolant pump coupled to the coolant circulation path and configured to control movement of the coolant in the coolant circulation path.

In Example 11, the subject matter of any one or any combination of Examples 3 to 10 may optionally be configured such that the implantable neurostimulator includes multiple implantable devices communicatively coupled to each other to increase a maximum frequency for the VHF stimulation waveform.

In Example 12, the subject matter of any one or any combination of Examples 1 to 11 may optionally be configured such that the one or more sensors include at least one temperature sensor configured to sense a temperature directly.

In Example 13, the subject matter of any one or any combination of Examples 1 to 12 may optionally be configured such that the one or more sensors include at least one physiological sensor configured to sense a physiological signal indicative of a temperature or a change of the temperature.

In Example 14, the subject matter of any one or any combination of Examples 1 to 13 may optionally be configured such that the stimulation parameter circuitry is configured to adjust the stimulation parameters using the temperature parameter to prevent the temperature or the temperature change from exceeding a specified threshold.

In Example 15, the subject matter of any one or any combination of Examples 1 to 14 may optionally be configured such that the stimulation parameter circuitry is configured to adjust the stimulation parameters using the temperature parameter to maintain the temperature or the temperature change within a specified range.

An example (e.g., “Example 16”) of a method for delivering neurostimulation to one or more regions of tissue in a patient is also provided. The method may include delivering the neurostimulation from a stimulation output circuit from a stimulation device, sensing a measure of tissue heating caused by the delivery of the neurostimulation using one or more sensors, determining a temperature parameter representing a temperature or a temperature change using the received measure of tissue heating, adjusting the stimulation parameters using the temperature parameter, and adjusting the stimulation parameters to limit a dosage based on an operational capability of the implantable neurostimulator and a safety limit related to tissue heating. The dosage may include an amount of at least one of an electrical energy or electrical charge injected in to the tissue by the delivery of the neurostimulation over a specified duration.

In Example 17, the subject matter of delivering the neurostimulation as found in Example 16 may optionally include delivering the neurostimulation using a very-high-frequency (VHF) stimulation waveform. The VHF stimulation waveform has a frequency of at least 100 kHz.

In Example 18, the subject matter of delivering the neurostimulation as found in Example 16 may optionally include delivering the neurostimulation using an implantable neurostimulator.

In Example 19, the subject matter of Example 18 may optionally further include providing one or more heat dissipators, providing the implantable neurostimulator with a thermal management device configured to be coupled to the one or more heat dissipators, providing a thermal conductive lead having a proximal end configured to be coupled to the thermal management device and a distal end configured to be placed in or about a region of the one or more regions of tissue, and providing a thermal conductive path using the thermal management device and the thermal conductive lead for a portion of a thermal energy causing the tissue heating to be dissipated through at least the one or more heat dissipators.

In Example 20, the subject matter of providing the one or more heat dissipators as found in Example 19 may optionally include providing at least one implantable heat dissipator configured to be coupled to the implantable neurostimulator.

In Example 21, the subject matter of providing the one or more heat dissipators as found in any one or any combination of Examples 19 and 20 may optionally include using a portion of the implantable neurostimulator as a heat dissipator of the one or more heat dissipators.

In Example 22, the subject matter of any one or any combination of Examples 19 to 21 may optionally further include connecting one or more implantable leads to the implantable neurostimulator. The one or more implantable leads each include a proximal end configured to be coupled, a distal end configured to be placed in or about a region of the one or more regions of tissue, and a plurality of electrodes at the distal end. At least one of the one or more implantable leads is configured to be the thermal conductive lead. The one or more sensors are each incorporated into the distal end of a lead of the one or more implantable leads. The subject matter of delivering the neurostimulation as found in any one or any combination of Examples 19 to 21 may optionally include delivering the neurostimulation using one or more electrodes selected from the plurality of electrodes at the distal end of each lead of the one or more implantable leads.

In Example 23, the subject matter of any one or any combination of Examples 19 to 22 may optionally further include forming a coolant circulation path in the one or more heat dissipators, the thermal management device, and the thermal conductive lead. The subject matter of providing the implantable stimulator with the thermal management device as found in any one or any combination of Examples 19 to 22 may optionally further include providing a thermal management device including a coolant reservoir coupled to the coolant circulation path and configured to store a coolant and a coolant pump coupled to the coolant circulation path and configured to control movement of the coolant in the coolant circulation path.

In Example 24, the subject matter of delivering the neurostimulation as found any one or any combination of Examples 18 to 23 may optionally include delivering the neurostimulation from multiple implantable devices communicatively coupled to each other to increase a maximum frequency for the VHF stimulation waveform.

An example (e.g., “Example 25”) of a non-transitory computer-readable storage medium including instructions, which when executed by a system, cause the system to perform a method for delivering neurostimulation to one or more regions of tissue in a patient is also provided. The method may include delivering the neurostimulation from a stimulation output circuit, receiving a measure of tissue heating caused by the delivery of the neurostimulation (the measure of tissue heating sensed using one or more sensors), determining a temperature parameter representing a temperature or a temperature change using the received measure of tissue heating, adjusting the stimulation parameters using the temperature parameter, and adjusting the stimulation parameters to limit a dosage based on an operational capability of the implantable neurostimulator and a safety limit related to tissue heating. The dosage may include an amount of at least one of an electrical energy or electrical charge injected in to the tissue by the delivery of the neurostimulation over a specified duration.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a neurostimulation system.

FIG. 2 illustrates an embodiment of a stimulation device and a lead system, such as may be implemented in the neurostimulation system of FIG. 1.

FIG. 3 illustrates an embodiment of a programming device, such as may be implemented in the neurostimulation system of FIG. 1.

FIG. 4 illustrates an embodiment of an implantable pulse generator (IPG) and an implantable lead system, such as an example implementation of the stimulation device and lead system of FIG. 2.

FIG. 5 illustrates an embodiment of an IPG and an implantable lead system, such as the IPG and lead system of FIG. 4, arranged to provide neurostimulation to a patient.

FIG. 6 illustrates an embodiment of portions of a neurostimulation system.

FIG. 7 illustrates an embodiment of an implantable stimulator and one or more leads of an implantable neurostimulation system, such as the implantable neurostimulation system of FIG. 6.

FIG. 8 illustrates an embodiment of an external programming device of an implantable neurostimulation system, such as the implantable neurostimulation system of FIG. 6.

FIG. 9 illustrates an embodiment of a system for delivering very-high-frequency (VHF) neurostimulation.

FIG. 10 illustrates an embodiment of an implantable system for VHF neurostimulation, including an implantable stimulator coupled to at least one cooling lead and a heat dissipator.

FIG. 11 illustrates an embodiment of an implantable system for VHF neurostimulation including multiple stimulators.

FIG. 12 illustrates an embodiment of an implantable stimulator of an implantable system for VHF neurostimulation, such as the implantable system of FIG. 10.

FIG. 13 illustrates an embodiment of a stimulation programming circuit of a programming device of an implantable system for VHF neurostimulation, such as the implantable system of FIG. 10.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, a method and system for delivering very-high-frequency neurostimulation therapy to a patient. As used in this document, a “very-high-frequency” (“VHF”) stimulation, also referred to as “very-high-rate stimulation”, “ultra-high-frequency (UHF) stimulation” or “ultra-high-rate stimulation”, may use a stimulation waveform having a frequency of about or above 100 kHz (e.g., a train of pulses delivered at 100 thousand or more pulses per second). VHF stimulation may also refer to stimulation using a frequency range of within which the mechanisms of interaction with biological targets, especially neural targets, are different from stimulation at lower frequencies. Such frequency range can start at approximately 500 to 1000 Hz. In various embodiments, the VHF neurostimulation can modulate neurological conditions and/or other physiological conditions of the patient though the neural system with or without first directly modulating neural firing. It is understood that the VHF neurostimulation can produce therapeutic effects through mechanisms of action that are different from those in lower-frequency neurostimulation. The VHF neurostimulation therapy can be delivered from a neurostimulation system and controlled using stimulation parameters. In various embodiments, the neuromodulation system can include an implantable device configured to deliver neurostimulation (also referred to as neuromodulation) therapies, such as deep brain stimulation (DBS), spinal cord stimulation (SCS), sacral nerve stimulation (SNS), peripheral nerve stimulation (PNS), and vagus nerve stimulation (VNS, including stimulation of main and branch nerves in neck and/or thoracic regions), and one or more external devices configured to program the implantable device for its operations and monitor the performance of the implantable device.

Various issues raised by VHF neurostimulation that are material to its efficacious and safe application include tissue heating, neuroanatomic effects (e.g., effects on cell membranes/gate and ionic concentrations, neurotransmission, or blood brain barrier), and cellular effects (e.g., homeostasis, repair, or division). The present subject matter can control delivery of VHF neurostimulation while allowing for management of such issues, for example by providing various ways for preventing the neurostimulation system from delivering excessive energy that cause unacceptable level of tissue heating and/or other unwanted effects. While neurostimulation using electrical pulses as the stimuli is specifically discussed as an example, the present subject matter can be applied to neurostimulation using any form of stimulation energy, including but not limited to any electrical and/or magnetic energy used as stimuli having VHF components.

Various embodiments include delivering the neurostimulation according to a pattern of neurostimulation pulses. The pattern of neurostimulation pulses can include a single VHF pulse rate or multiple pulse rate including VHF and lower frequencies (e.g., around 1 kHz). The pattern of neurostimulation pulses can include one or more individually programmable waveforms. Each waveform can be charge balanced, with a programmable recharge scheme selected from various recharge phase schemes (e.g., active, passive, single recharge phase following a burst of pulses, and the like).

A neurostimulation system according to the present subject matter can include one or more implantable leads, an implantable stimulation device, and a programming device. The one or more implantable leads can be used for delivering the neurostimulation pulses and for thermal management. For example, a lead can be used to sense a temperature at or near a stimulation site in the patient for applying thermal management rules for controlling (e.g., minimizing) tissue heating at the stimulation site caused by the neurostimulation (e.g., feedback control of stimulation parameters using sensed temperature). In another example, a lead can be used to transfer heat from the stimulation site to a distant and/or larger surface area or volume for dissipation. To meet requirements for VHF neurostimulation, the implantable stimulation device can include low-power, high-speed electronics providing, for example, a clock signal at a frequency sufficiently higher for digitally timing the neurostimulation pulses delivered at VHF (e.g., a clock frequency of at least twice of the pulse frequency), very short digital signal rising/falling times, and a stable and sufficient power supply for a stimulation output circuit to generate the neurostimulation pulses at VHF with required amplitude. The programming device can include a user interface including a composer capable of composing the pattern of neurostimulation pulses while managing the issues material to VHF neurostimulation, such as by enforcing programming rules governing parameter interlocks and preventing excessive energy or electric charges from being delivered to each stimulation site.

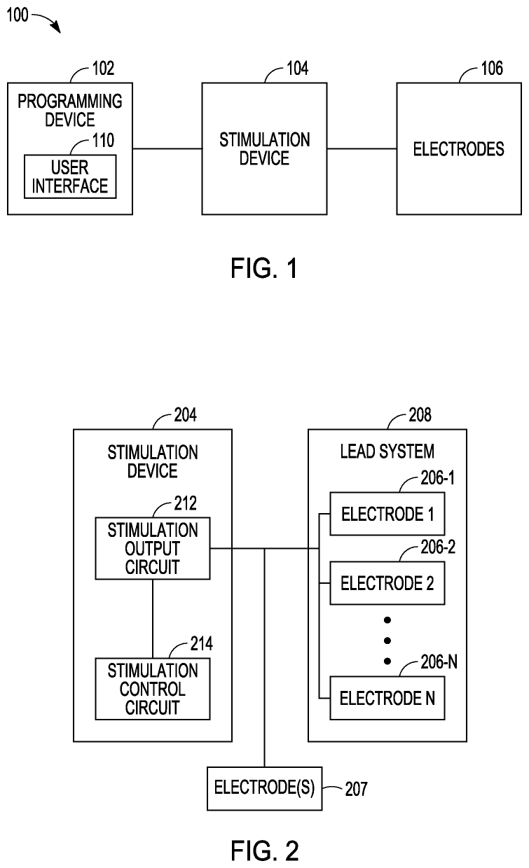

FIG. 1 illustrates an embodiment of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device via a wired or wireless link.

In this document, a “user” includes a physician or other clinician or caregiver who examiners and/or treats the patient using system 100; a “patient” includes a person who receives or is intended to receive neurostimulation delivered using system 100. In various embodiments, the patient can be allowed to adjust his or her treatment using system 100 to certain extent, such as by adjusting certain therapy parameters and entering feedback and clinical effect information.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for DBS applications. Such DBS configuration includes various features that may simplify the task of the user in programming stimulation device 104 for delivering DBS to the patient, such as the features discussed in this document.

FIG. 2 illustrates an embodiment of a stimulation device 204 and a lead system 208, such as may be implemented in neurostimulation system 100. Stimulation device 204 represents an embodiment of stimulation device 104 and includes a stimulation output circuit 212 and a stimulation control circuit 214. Stimulation output circuit 212 produces and delivers neurostimulation pulses. Stimulation control circuit 214 controls the delivery of the neurostimulation pulses from stimulation output circuit 212 using the plurality of stimulation parameters, which specifies a pattern of the neurostimulation pulses. Lead system 208 includes one or more leads each configured to be electrically connected to stimulation device 204 and a plurality of electrodes 206 distributed in the one or more leads. The plurality of electrodes 206 includes electrode 206-1, electrode 206-2, . . . electrode 206-N, each a single electrically conductive contact providing for an electrical interface between stimulation output circuit 212 and tissue of the patient, where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes. In various embodiments, one or more additional electrodes 207 (each of which may be referred to as a reference electrode) can be electrically connected to stimulation device 204, such as one or more electrodes each being a portion of or otherwise incorporated onto a housing of stimulation device 204. Monopolar stimulation uses a monopolar electrode configuration with one or more electrodes selected from electrodes 206 and at least one electrode from electrode(s) 207. Bipolar stimulation uses a bipolar electrode configuration with two electrodes selected from electrodes 206 and none electrode(s) 207. Multipolar stimulation uses a multipolar electrode configuration with multiple (two or more) electrodes selected from electrodes 206 and none of electrode(s) 207.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes.

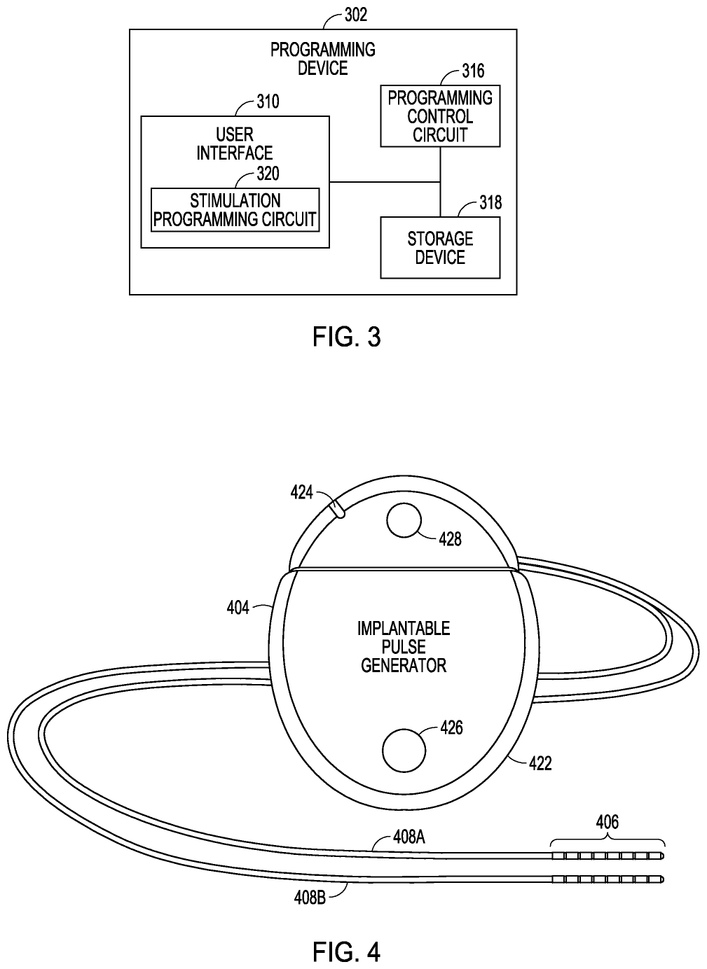

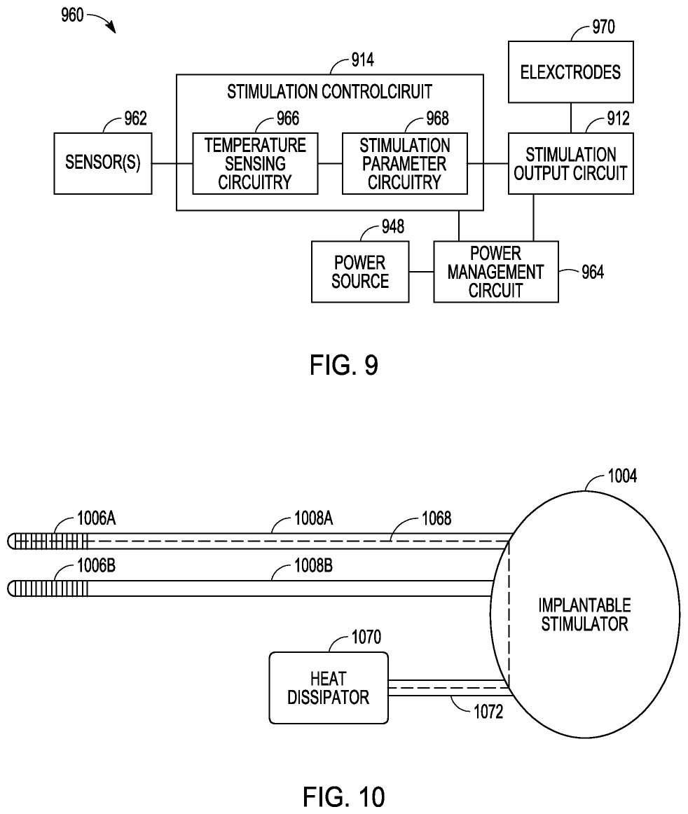

FIG. 3 illustrates an embodiment of a programming device 302, such as may be implemented in neurostimulation system 100. Programming device 302 represents an embodiment of programming device 102 and includes a storage device 318, a programming control circuit 316, and a user interface 310. Programming control circuit 316 generates the plurality of stimulation parameters that controls the delivery of the neurostimulation pulses according to a specified stimulation configuration that can define, for example, stimulation waveform and electrode configuration. User interface 310 represents an embodiment of user interface 110 and includes a stimulation programming circuit 320. Storage device 318 stores information used by programming control circuit 316 and stimulation programming circuit 320, such as information about a stimulation device that relates the stimulation configuration to the plurality of stimulation parameters and information relating the stimulation configuration to a volume of activation in the patient. In various embodiments, stimulation programming circuit 320 can be configured to support one or more functions allowing for programming of stimulation devices, such as stimulation device 104 including its various embodiments as discussed in this document, to control delivery of neurostimulation pulses using stimulation parameters with parameter dithering according to the present subject matter, as further discussed below with reference to FIGS. 9-13.

In various embodiments, user interface 310 can allow for definition of a pattern of neurostimulation pulses for delivery during a neurostimulation therapy session by creating and/or adjusting one or more stimulation waveforms using a graphical method. The definition can also include definition of one or more stimulation fields each associated with one or more pulses in the pattern of neurostimulation pulses. As used in this document, a “stimulation configuration” can include the pattern of neurostimulation pulses including the one or more stimulation fields, or at least various aspects or parameters of the pattern of neurostimulation pulses including the one or more stimulation fields. In various embodiments, user interface 310 includes a GUI that allows the user to define the pattern of neurostimulation pulses and perform other functions using graphical methods. In this document, “neurostimulation programming” can include the definition of the one or more stimulation waveforms, including the definition of one or more stimulation fields.

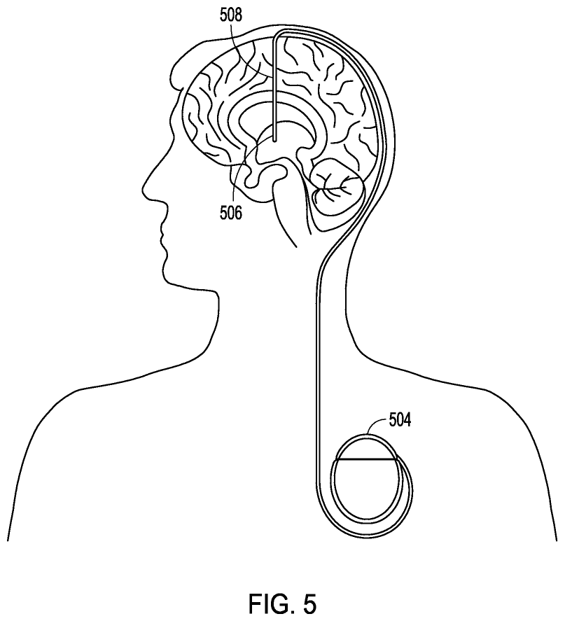

FIG. 4 illustrates an embodiment of an implantable pulse generator (IPG) 404 and an implantable lead system 408. IPG 404 represents an example implementation of stimulation device 204. Lead system 408 represents an example implementation of lead system 208. As illustrated in FIG. 4, IPG 404 that can be coupled to implantable leads 408A and 408B at a proximal end of each lead. The distal end of each lead includes electrical contacts or electrodes 406 for contacting a tissue site targeted for electrical neurostimulation. As illustrated in FIG. 1, leads 408A and 408B each include 8 electrodes 406 at the distal end. The number and arrangement of leads 408A and 408B and electrodes 406 as shown in FIG. 1 are only an example, and other numbers and arrangements are possible. In various embodiments, the electrodes are ring electrodes. The implantable leads and electrodes may be configured by shape and size to provide electrical neurostimulation energy to a neuronal target included in the subject's brain, or configured to provide electrical neurostimulation energy to a nerve cell target included in the subject's spinal cord.

FIG. 5 illustrates an embodiment of an IPG 504 and an implantable lead system 508 arranged to provide neurostimulation to a patient. An example of IPG 504 includes IPG 404. An example of lead system 508 includes one or more of leads 408A and 408B. In the illustrated embodiment, implantable lead system 508 is arranged to provide Deep Brain Stimulation (DBS) to a patient, with the stimulation target being neuronal tissue in a subdivision of the thalamus of the patient's brain. Other examples of DBS targets include neuronal tissue of the globus pallidus (GPi), the subthalamic nucleus (STN), the pedunculopontine nucleus (PPN), substantia nigra pars reticulate (SNr), globus pallidus externus (GPe), medial forebrain bundle (MFB), periaquaductal gray (PAG), periventricular gray (PVG), habenula, subgenual cingulate cortex, ventral intermediate nucleus (VIM) of the thalamus, anterior nucleus (AN) or other nuclei of the thalamus, zona incerta, ventral capsule, ventral striatum, nucleus accumbens, and other white matter tracts connecting these and other structures. DBS is discussed as an example for the present subject matter, which can be applied to other brain stimulation (e.g., cortical stimulation) and other neurostimulation therapies.

Returning to FIG. 4, the IPG 404 can include a hermetically-sealed IPG case 422 to house the electronic circuitry of IPG 404. IPG 404 can include an electrode 426 formed on IPG case 422. In some embodiments, IPG case 422 can be used as electrode 426. IPG 404 can include an IPG header 424 for coupling the proximal ends of leads 408A and 408B. IPG header 424 may optionally also include an electrode 428. Electrodes 426 and/or 428 represent embodiments of electrode(s) 207 and may each be referred to as a reference electrode. Neurostimulation energy can be delivered in a monopolar (also referred to as unipolar) mode using electrode 426 or electrode 428 and one or more electrodes selected from electrodes 406. Neurostimulation energy can be delivered in a bipolar mode using a pair of electrodes of the same lead (lead 408A or lead 408B). Neurostimulation energy can be delivered in an extended bipolar mode using one or more electrodes of a lead (e.g., one or more electrodes of lead 408A) and one or more electrodes of a different lead (e.g., one or more electrodes of lead 408B).

The electronic circuitry of IPG 404 can include a control circuit that controls delivery of the neurostimulation energy. The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

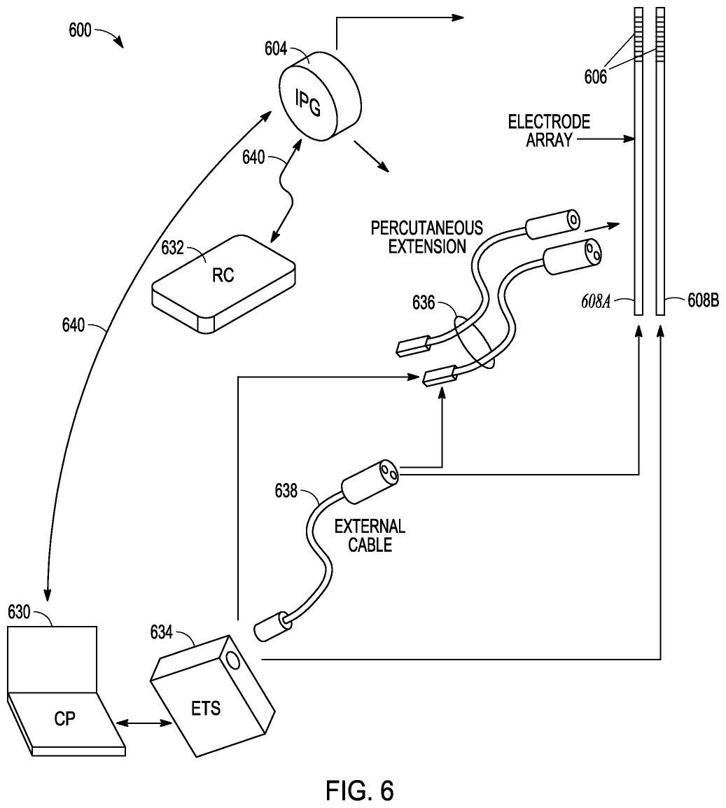

FIG. 6 illustrates an embodiment of portions of a neurostimulation system 600. System 600 includes an IPG 604, implantable neurostimulation leads 608A and 608B, an external remote controller (RC) 632, a clinician's programmer (CP) 630, and an external trial stimulator (ETS) 634. IPG 404 may be electrically coupled to leads 608A and 608B directly or through percutaneous extension leads 636. ETS 634 may be electrically connectable to leads 608A and 608B via one or both of percutaneous extension leads 636 and/or external cable 638. System 600 represents an embodiment of system 100, with IPG 604 representing an embodiment of stimulation device 104, electrodes 606 of leads 608A and 608B representing electrodes 106, and CP 630, RC 632, and ETS 634 collectively representing programming device 102.

ETS 634 may be standalone or incorporated into CP 630. ETS 634 may have similar pulse generation circuitry as IPG 604 to deliver neurostimulation energy according to specified modulation parameters as discussed above. ETS 634 is an external device that is typically used as a preliminary stimulator after leads 408A and 408B have been implanted and used prior to stimulation with IPG 604 to test the patient's responsiveness to the stimulation that is to be provided by IPG 604. Because ETS 634 is external it may be more easily configurable than IPG 604.

CP 630 can configure the neurostimulation provided by ETS 634. If ETS 634 is not integrated into CP 630, CP 630 may communicate with ETS 634 using a wired connection (e.g., over a USB link) or by wireless telemetry using a wireless communications link 640. CP 630 also communicates with IPG 604 using a wireless communications link 640.

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication. IPG 604 can include the first coil and a communication circuit. CP 630 can include or otherwise electrically connected to the second coil such as in the form of a wand that can be place near IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2π, where λ is the wavelength of the transmitted electromagnetic energy. In one example, a communication range of an RF telemetry link is at least six feet but can be as long as allowed by the particular communication technology. RF antennas can be included, for example, in the header of IPG 604 and in the housing of CP 630, eliminating the need for a wand or other means of inductive coupling. An example is such an RF telemetry link is a Bluetooth® wireless link.

CP 630 can be used to set modulation parameters for the neurostimulation after IPG 604 has been implanted. This allows the neurostimulation to be tuned if the requirements for the neurostimulation change after implantation. CP 630 can also upload information from IPG 604.

RC 632 also communicates with IPG 604 using a wireless link 340. RC 632 may be a communication device used by the user or given to the patient. RC 632 may have reduced programming capability compared to CP 630. This allows the user or patient to alter the neurostimulation therapy but does not allow the patient full control over the therapy. For example, the patient may be able to increase the amplitude of neurostimulation pulses or change the time that a preprogrammed stimulation pulse train is applied. RC 632 may be programmed by CP 630. CP 630 may communicate with the RC 632 using a wired or wireless communications link. In some embodiments, CP 630 is able to program RC 632 when remotely located from RC 632.

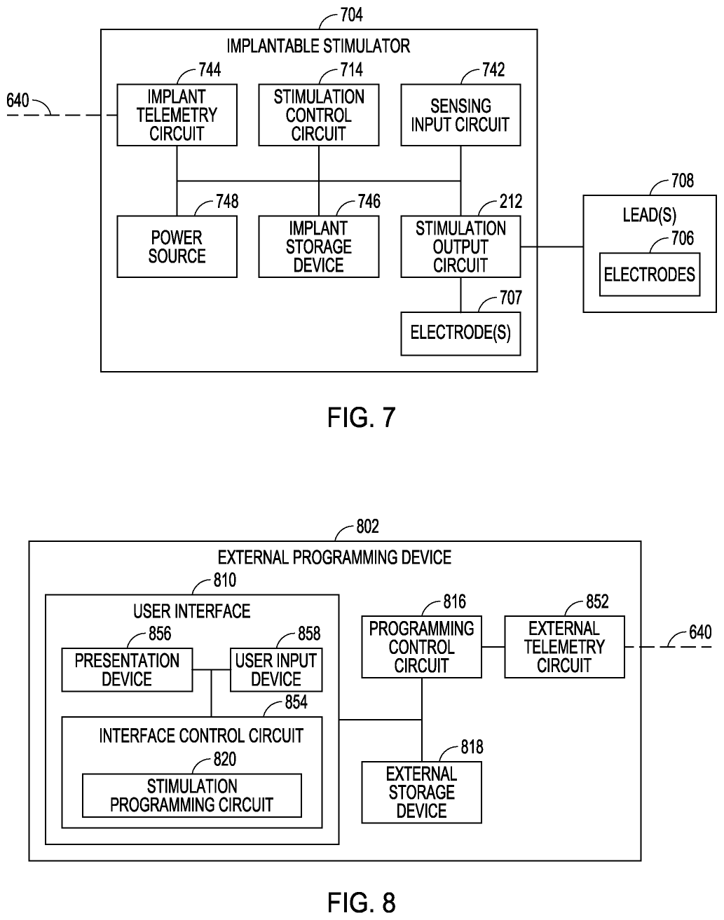

FIG. 7 illustrates an embodiment of implantable stimulator 704 and one or more leads 708 of an implantable neurostimulation system, such as implantable system 600. Implantable stimulator 704 represents an embodiment of stimulation device 104 or 204 and may be implemented, for example, as IPG 604. Lead(s) 708 represents an embodiment of lead system 208 and may be implemented, for example, as implantable leads 608A and 608B. Lead(s) 708 includes electrodes 706, which represents an embodiment of electrodes 106 or 206 and may be implemented as electrodes 606.

Implantable stimulator 704 may include a sensing input circuit (also known as a sensing circuit) 742 that provides the stimulator with a sensing capability, stimulation output circuit 212, a stimulation control circuit 714, an implant storage device 746, an implant telemetry circuit 744, a power source 748, and one or more electrodes 707. Sensing input circuit 742 senses one or more physiological signals for purposes of patient monitoring and/or feedback control of the neurostimulation. Examples of the one or more physiological signals include neural and other signals each indicative of a condition of the patient that is treated by the neurostimulation and/or a response of the patient to the delivery of the neurostimulation. Stimulation output circuit 212 is electrically connected to electrodes 706 through one or more leads 708 as well as electrodes 707, and delivers each of the neurostimulation pulses through a set of electrodes selected from electrodes 706 and electrode(s) 707. Stimulation control circuit 714 represents an embodiment of stimulation control circuit 214 and controls the delivery of the neurostimulation pulses using the plurality of stimulation parameters specifying the pattern of neurostimulation pulses. In one embodiment, stimulation control circuit 714 controls the delivery of the neurostimulation pulses using the one or more sensed physiological signals. Implant telemetry circuit 744 provides implantable stimulator 704 with wireless communication with another device such as CP 630 and RC 632, including receiving values of the plurality of stimulation parameters from the other device. Implant storage device 746 stores values of the plurality of stimulation parameters. Power source 748 provides implantable stimulator 704 with energy for its operation. In one embodiment, power source 748 includes a battery. In one embodiment, power source 748 includes a rechargeable battery and a battery charging circuit for charging the rechargeable battery. Implant telemetry circuit 744 may also function as a power receiver that receives power transmitted from an external device through an inductive couple. Electrode(s) 707 allow for delivery of the neurostimulation pulses in the monopolar mode. Examples of electrode(s) 707 include electrode 426 and electrode 418 in IPG 404 as illustrated in FIG. 4.

In one embodiment, implantable stimulator 704 is used as a master database. A patient implanted with implantable stimulator 704 (such as may be implemented as IPG 604) may therefore carry patient information needed for his or her medical care when such information is otherwise unavailable. Implant storage device 746 is configured to store such patient information. For example, the patient may be given a new RC 632 and/or travel to a new clinic where a new CP 630 is used to communicate with the device implanted in him or her. The new RC 632 and/or CP 630 can communicate with implantable stimulator 704 to retrieve the patient information stored in implant storage device 746 through implant telemetry circuit 744 and wireless communication link 640, and allow for any necessary adjustment of the operation of implantable stimulator 704 based on the retrieved patient information. In various embodiments, the patient information to be stored in implant storage device 746 may include, for example, positions of lead(s) 708 and electrodes 706 relative to the patient's anatomy (transformation for fusing computerized tomogram (CT) of post-operative lead placement to magnetic resonance imaging (MRI) of the brain), clinical effect map data, objective measurements using quantitative assessments of symptoms (for example using micro-electrode recording, accelerometers, and/or other sensors), and/or any other information considered important or useful for providing adequate care for the patient. In various embodiments, the patient information to be stored in implant storage device 746 may include data transmitted to implantable stimulator 704 for storage as part of the patient information and data acquired by implantable stimulator 704, such as by using sensing input circuit 742.

In various embodiments, sensing input circuit 742, stimulation output circuit 212, stimulation control circuit 714, implant telemetry circuit 744, implant storage device 746, and power source 748 are encapsulated in a hermetically sealed implantable housing or case, and electrode(s) 707 are formed or otherwise incorporated onto the case. In various embodiments, lead(s) 708 are implanted such that electrodes 706 are placed on and/or around one or more targets to which the neurostimulation pulses are to be delivered, while implantable stimulator 704 is subcutaneously implanted and connected to lead(s) 708 at the time of implantation.

FIG. 8 illustrates an embodiment of an external programming device 802 of an implantable neurostimulation system, such as system 600. External programming device 802 represents an embodiment of programming device 102 or 302, and may be implemented, for example, as CP 630 and/or RC 632. External programming device 802 includes an external telemetry circuit 852, an external storage device 818, a programming control circuit 816, and a user interface 810.

External telemetry circuit 852 provides external programming device 802 with wireless communication with another device such as implantable stimulator 704 via wireless communication link 640, including transmitting the plurality of stimulation parameters to implantable stimulator 704 and receiving information including the patient data from implantable stimulator 704. In one embodiment, external telemetry circuit 852 also transmits power to implantable stimulator 704 through an inductive couple.

In various embodiments, wireless communication link 640 can include an inductive telemetry link (near-field telemetry link) and/or a far-field telemetry link (RF telemetry link). For example, because DBS is often indicated for movement disorders which are assessed through patient activities, gait, balance, etc., allowing patient mobility during programming and assessment is useful. Therefore, when system 600 is intended for applications including DBS, wireless communication link 640 includes at least a far-field telemetry link that allows for communications between external programming device 802 and implantable stimulator 704 over a relative long distance, such as up to about 20 meters. External telemetry circuit 852 and implant telemetry circuit 744 each include an antenna and RF circuitry configured to support such wireless telemetry.

External storage device 818 stores one or more stimulation waveforms for delivery during a neurostimulation therapy session, such as a DBS therapy session, as well as various parameters and building blocks for defining one or more waveforms. The one or more stimulation waveforms may each be associated with one or more stimulation fields and represent a pattern of neurostimulation pulses to be delivered to the one or more stimulation field during the neurostimulation therapy session. In various embodiments, each of the one or more stimulation waveforms can be selected for modification by the user and/or for use in programming a stimulation device such as implantable stimulator 704 to deliver a therapy. In various embodiments, each waveform in the one or more stimulation waveforms is definable on a pulse-by-pulse basis, and external storage device 818 may include a pulse library that stores one or more individually definable pulse waveforms each defining a pulse type of one or more pulse types. External storage device 818 also stores one or more individually definable stimulation fields. Each waveform in the one or more stimulation waveforms is associated with at least one field of the one or more individually definable stimulation fields. Each field of the one or more individually definable stimulation fields is defined by a set of electrodes through a neurostimulation pulse is delivered. In various embodiments, each field of the one or more individually definable fields is defined by the set of electrodes through which the neurostimulation pulse is delivered and a current distribution of the neurostimulation pulse over the set of electrodes. In one embodiment, the current distribution is defined by assigning a fraction of an overall pulse amplitude to each electrode of the set of electrodes. Such definition of the current distribution may be referred to as “fractionalization” in this document. In another embodiment, the current distribution is defined by assigning an amplitude value to each electrode of the set of electrodes. For example, the set of electrodes may include 2 electrodes used as the anode and an electrode as the cathode for delivering a neurostimulation pulse having a pulse amplitude of 4 mA. The current distribution over the 2 electrodes used as the anode needs to be defined. In one embodiment, a percentage of the pulse amplitude is assigned to each of the 2 electrodes, such as 75% assigned to electrode 1 and 25% to electrode 2. In another embodiment, an amplitude value is assigned to each of the 2 electrodes, such as 3 mA assigned to electrode 1 and 1 mA to electrode 2. Control of the current in terms of percentages allows precise and consistent distribution of the current between electrodes even as the pulse amplitude is adjusted. It is suited for thinking about the problem as steering a stimulation locus, and stimulation changes on multiple contacts simultaneously to move the locus while holding the stimulation amount constant. Control and displaying the total current through each electrode in terms of absolute values (e.g., mA) allows precise dosing of current through each specific electrode. It is suited for changing the current one contact at a time (and allows the user to do so) to shape the stimulation like a piece of clay (pushing/pulling one spot at a time).

Programming control circuit 816 represents an embodiment of programming control circuit 316 and generates the plurality of stimulation parameters, which is to be transmitted to implantable stimulator 704, based on a specified stimulation configuration (e.g., the pattern of neurostimulation pulses as defined by one or more stimulation waveforms and one or more stimulation fields, or at least certain aspects of the pattern). The stimulation configuration may be created and/or adjusted by the user using user interface 810 and stored in external storage device 818. In various embodiments, programming control circuit 816 can check values of the plurality of stimulation parameters against safety rules to limit these values within constraints of the safety rules. In one embodiment, the safety rules are heuristic rules.

User interface 810 represents an embodiment of user interface 310 and allows the user to define the pattern of neurostimulation pulses and perform various other monitoring and programming tasks. User interface 810 includes a display screen 856, a user input device 858, and an interface control circuit 854. Display screen 856 may include any type of interactive or non-interactive screens, and user input device 858 may include any type of user input devices that supports the various functions discussed in this document, such as touchscreen, keyboard, keypad, touchpad, trackball, joystick, and mouse. In one embodiment, user interface 810 includes a GUI. The GUI may also allow the user to perform any functions discussed in this document where graphical presentation and/or editing are suitable as may be appreciated by those skilled in the art.

Interface control circuit 854 controls the operation of user interface 810 including responding to various inputs received by user input device 858 and defining the one or more stimulation waveforms. Interface control circuit 854 includes a stimulation programming circuit 820.

In various embodiments, external programming device 802 can have operation modes including a composition mode and a real-time programming mode. Under the composition mode (also known as the pulse pattern composition mode), user interface 810 is activated, while programming control circuit 816 is inactivated. Programming control circuit 816 does not dynamically updates values of the plurality of stimulation parameters in response to any change in the one or more stimulation waveforms. Under the real-time programming mode, both user interface 810 and programming control circuit 816 are activated. In some embodiments, one or more additional user interfaces (e.g., in one or more additional programming devices) are used in an online (e.g., real-time) programming mode and/or an offline (e.g., composing) programming mode. Programming control circuit 816 dynamically updates values of the plurality of stimulation parameters in response to changes in the set of one or more stimulation waveforms, and transmits the plurality of stimulation parameters with the updated values to implantable stimulator 704.

Stimulation programming circuit 820 can determine the stimulation configuration, such as the pattern of neurostimulation pulses as defined by one or more stimulation waveforms and one or more stimulation fields, that is suitable for VHF neurostimulation. In various embodiments, stimulation programming circuit 820 operates with presentation device 856 and user input device 858 to compose the pattern of neurostimulation pulses automatically, semi-automatically with various degree of participation from the user, or manually by allowing the user to create and/or edit any adjustable components of the pattern. Neurostimulation pulses as a form of VHF neurostimulation is discussed in this document as an example, rather than a restriction, of the present subject matter. Examples other forms of neurostimulation include other VHF waveforms such as sinusoidal wave or digitized sinusoidal wave with high resolution.

The pattern of neurostimulation pulses can include one or more stimulation periods and one or more non-stimulation periods. The one or more stimulation periods (also referred to as blocks, stimulation blocks, and the like) are each a period during which the neurostimulation is delivered. The neurostimulation delivered during a stimulation period can include bursts of neurostimulation pulses at a VHF pulse rate (e.g., 100 kHz or above) delivered at a burst rate (number of bursts per second) (e.g., 500 Hz, 100 Hz, 50 Hz, or 10 Hz). At the pulse rate of 100 kHz (corresponding to the period of 10 μs, the pulse width is limited to less than 10 μs (without immediate recharge phase for charge balancing). The one or more non-stimulation periods (also referred to as delays, delay blocks, silence blocks, and the like) are each a period during which no neurostimulation is delivered. In various embodiments, a non-stimulation period can follow a stimulation period for charge balancing and/or thermal management purposes.

The pattern of neurostimulation pulses can include one or more stimulation periods can include one or more cooling periods for safety (e.g., prevention from tissue over-heating and/or other unwanted effects). The one or more stimulation periods can each be a non-stimulation period or a stimulation period. An example of a cooling period includes a non-stimulation period following a stimulation period to stop heating caused by the neurostimulation delivered during the stimulation period. Another example of a cooling period includes a stimulation period during which the neurostimulation is delivered at a low energy level (e.g., low frequency and/or low pulse amplitude) determined to cause an insignificant amount of tissue heating, for example following another stimulation period during which a significant amount of tissue heating is caused. Yet another example of a cooling period includes a stimulation period during which the neurostimulation is delivered to a tissue region for cooling a different tissue region (where the tissue has been heated by the neurostimulation). Still another example of a cooling period includes a stimulation period during which the neurostimulation is delivered in order to cause, enable, or cooling of a previously heated tissue region.

The pattern of neurostimulation pulses can include one or more stimulation periods can include interleaved VHF neurostimulation. For example, the pattern can include neurostimulation pulses at VHF (e.g., 100 kHz or above) interleaved with neurostimulation pulses at lower frequencies (e.g., 1.2 kHz or below).

The pattern of neurostimulation pulses can include one or more stimulation periods can include multiple stimulation waveforms. For example, the pattern of neurostimulation pulses can include pulse sequences each using a different stimulation waveform. The multiple stimulation waveforms can be time for simultaneous delivery (e.g., to multiple stimulation sites in the patient) or alternating delivery (e.g., to the same stimulation site). Examples of the multiple stimulation waveforms include multiple VHF stimulation waveforms, a combination of VHF and non-VHF stimulation waveforms, bursts of VHF pulses delivered at a VHF burst rate, and bursts of VHF pulses delivered at a non-VHF burst rate.

The pattern of neurostimulation pulses can include stimulation and recharge phases for charge balancing. Various recharge schemes including active and/or passive recharge phases can be applied to the pattern of neurostimulation for the purpose of charge balancing. A recharge scheme (defining how recharge phases are arranged relative to stimulation phases in a stimulation waveform) can be selected for reduction of power consumption of the stimulation device (e.g., a battery-powered implantable neurostimulator), for additional therapeutic benefits, and/or for other effects. The recharge schemes can include symmetric active recharge, where the pulse widths and amplitudes of opposite polarities are equal, and asymmetric active recharge, particularly where the recharge phase has a lower amplitude and longer pulse width. The recharge schemes can include pulse phase polarities designed to bring a particular circuit property toward or to zero, such as an interface potential or a total net charge delivered. Active and passive recharge pulses can be interspersed, and charge-recovery schemes can seek a goal such as zero-balance in a pairwise fashion, or given some longer series of pulses or pulse phases (e.g., every 3, 7, 100, or other number of pulses or phases). A particular recharge scheme may allow for the neurostimulation to achieve an intended outcome with fewer pulses and/or reduced pulse amplitude. Periods of non-stimulation during recharge may be tolerable or, in some cases, desirable for therapeutic effects (e.g., neural plasticity induced long-lasting reduction of symptoms). Stimulation (e.g., cathodic) and recharge (e.g., anodic) phases of a stimulation waveform may activate different neural elements, so both phases may contribute to the overall therapeutic effect. Recharge periods can be designed to afford interruption from interleaved stimulation. Recharge schemes can be designed to afford superposition of additional channels of stimulation. Recharge can be designed to afford at least either or both of interleaving and superimposing stimulation.

Examples of the various recharge schemes include:
a burst of neurostimulation pulses with only stimulation phases, followed by a long active or passive recharge pulse (e.g., 100 cathodic stimulation pulses at followed by a long anodic recharge pulse, or 100 anodic stimulation pulses at followed by a long cathodic recharge pulse);
a burst of neurostimulation pulses with only stimulation phases, followed a burst of symmetric active recharge pulses (e.g., 100 cathodic stimulation pulses followed by 100 anodic recharge pulses, or 100 anodic stimulation pulses followed by 100 cathodic recharge pulses);
a burst of neurostimulation pulses with only stimulation phases preceded by a recharge pulse (“pre-pulsing”) and followed by another recharge pulse (“post-pulsing”); and
a burst of neurostimulation pulses at VHF with only stimulation phases, followed by an active recharge pulse and a passive recharge pulse (the active recharge pulse and then the passive recharge pulse, or the passive recharge pulse and then the active recharge pulse).
   Various examples of recharging schemes and timing are discussed in U.S. Pat. No. 10,994,143 B2, assigned to Boston Scientific Neuromodulation Corporation, which is incorporated by reference herein in its entirety.

FIG. 9 illustrates an embodiment of a system 960 for delivering VHF neurostimulation to one or more regions of tissue in a patient. System 960 can include one or more sensors 962, a stimulation output circuit 912, electrodes 970, a power source 948, a power management circuit 964, and a stimulation control circuit 914. Sensor(s) 962 can be placed in the patient to sense a measure of tissue heating caused by the delivery of the neurostimulation. Stimulation output circuit 912 can deliver the neurostimulation using a VHF stimulation waveform to the patient. The VHF stimulation waveform can have a frequency of 100 kHz or above. Power source 948 can include one or more batteries. Power management circuit 964 can provide stimulation output circuit 912 with a power supply signal and to maintain a specified voltage level for the power supply signal during the delivery of the neurostimulation using the VHF stimulation waveform. Stimulation control circuit 914 can control the delivery of the neurostimulation using stimulation parameters and can include temperature sensing circuitry 966 and stimulation parameter circuitry 968. Temperature sensing circuitry 966 can receive the measure of tissue heating sensed by sensor(s) 962 and determine a temperature parameter representing a temperature or a temperature change using the received measure of tissue heating. Stimulation parameter circuitry 968 can adjust the stimulation parameters using the temperature parameter and adjust the stimulation parameters to limit a dosage based on an operational capability of stimulation output circuit 912 and power management circuit 964 and a safety limit related to tissue heating. The dosage can be specified by an amount or rate of an electrical energy and/or an amount of electrical charge injected in to the tissue of the patient by the delivery of the neurostimulation over a specified duration.

System 960 can in implemented as an implantable medical device. For example, implantable stimulator 704 and lead(s) 708 can be configured to include system 960. Sensor(s) 962 can be incorporated into implantable stimulator 704 and/or lead(s) 708 and/or can be stand-alone sense(s) communicatively coupled to implantable stimulator 704. Stimulation output circuit 212 can be configured to include stimulation output circuit 912, which can deliver the VHF neurostimulation. Electrodes 970 can include electrodes selected from electrodes 706 and 707. Power source 748 can be configured to include power source 948, which can provide sufficient power for the VHF neurostimulation. Stimulation control circuit 714 can be configured to include power management circuit 964 and stimulation control circuit 914.

Sensor(s) 962 can determine (e.g., measure or estimate) a temperature (absolute temperature, e.g., 40° C.) or a temperature change (relative temperature, e.g., 4° C. increase) from physical phenomena such as changes in resistance, impedance, optical, and/or chemical properties of tissue or fluid of the patient. Additionally, absolute or relative measures of temperature can be determined or inferred from measurements of biological properties, such as firing rates, conduction velocities, and the like. In various embodiments, sensor(s) 962 can include one or more electrodes of the lead, such as a lead of lead(s) 708. Electrical properties of the one or more electrodes can be measured to indicate the temperature or a temperature change. Sensor(s) 962 can include one or more sensing elements other than the electrodes, such as thermocouple, thermistor, interferometry sensor, and the like. Sensor(s) 962 can include one or more physiological sensors each sensing a signal that can be used as a surrogate of the temperature. For example, temperature can be inferred from blood flow measurements, for example using near infrared imaging or doppler (transcranial or implantable) sensor.

FIG. 10 illustrates an embodiment of an implantable system for VHF neurostimulation, including an implantable stimulator 1004 coupled to at least one cooling lead 1008A and a heat dissipator 1070. In the illustrated embodiment, another lead 1008B is also coupled to implantable stimulator 1004. The system as shown in FIG. 10 is to illustrate a thermal management structure by way of example, but not by way of restriction. Implantable stimulator 1004 and leads 1008A-B can represent an example of implantable stimulator 704 and lead(s) 708 configured to include system 960. Lead 1008A is a thermal conductive lead (also referred to as a “cooling lead” for use with implantable stimulator 1004 and/or a heat dissipator 1070 for transferring heat generated by the VHF neurostimulation away from the stimulation site for dissipation over a larger tissue volume. Lead 1008A can be an implantable lead as discussed above (e.g., any one of leads 408A, 408B, 608A, 608B, and 708) that include a thermal conductive conduit 1068. Thus, lead 1008A can include electrodes 1006A for stimulation and/or sensing and thermal conductive conduit 1068 for absorbing a portion of thermal energy created by the VHF stimulation and transfer the absorbed portion of the thermal energy to implantable stimulator 1004 and/or heat dissipator 1070. In one embodiment, thermal conductive conduit 1068 can be a wire, a cable, a heat-pipe, or the like made of a material having high thermal conductivity. In another embodiment, thermal conductive conduit 1068 includes a coolant conduit allowing for circulation of a coolant to absorb the thermal energy from tissue adjacent the lead and transfer the absorbed thermal energy to implantable stimulator 1004 and/or heat dissipator 1070. The thermal energy can be dissipated using the housing of implantable stimulator 1004 and/or heat dissipator 1070. Lead 1008B can be an implantable lead as discussed above (e.g., any one of leads 408A, 408B, 608A, 608B, and 708) with electrodes 1006B for stimulation and/or sensing.

In various embodiments, the implantable system for VHF neurostimulation can include implantable stimulator 1004, one or more cooling leads (e.g., lead 1008A) and optionally non-cooling lead (e.g., lead 1008B), and optionally heat dissipator 1070. Cooling of a tissue region targeted by the VHF neurostimulation can be achieved by providing greater thermal conductivity at or near that tissue region on to spread or carry away the thermal energy from that region to a greater surface area or volume for safe dissipation of the thermal energy. Depending on the amount of tissue heating anticipated, the housing of implantable stimulator 1004 and/or one or more implantable heat dissipator (e.g., heat dissipator 1070) can be used for the safe dissipation of the thermal energy. Optionally, the leads can each be designed with additional heat dissipation properties or advantages.

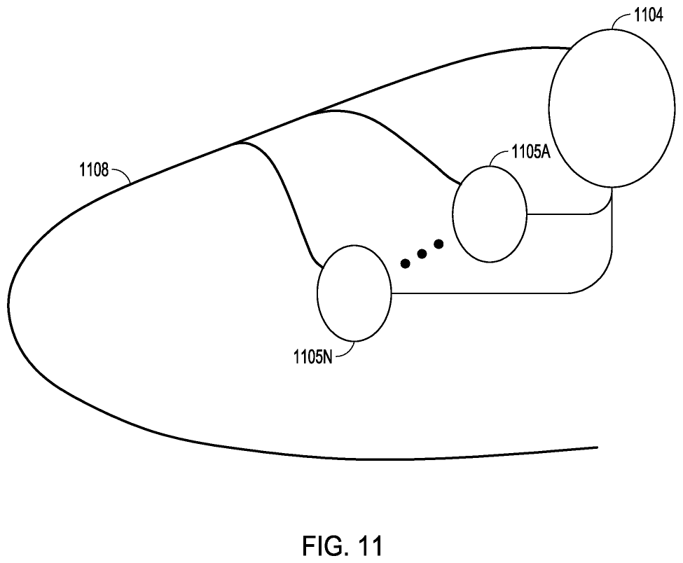

FIG. 11 illustrates an embodiment of an implantable system for VHF neurostimulation including multiple stimulators. In the illustrated embodiment, the implantable system includes an implantable stimulator 1104 coupled to one or more additional implantable stimulation units 1105A-N (N=1, 2, . . . ) that multiplies pulses to be delivered through a lead or lead system 1108. Implantable stimulator 1104 and lead or lead system 1108 can represent an example of implantable stimulator 704 and lead(s) 708 configured to include system 960. In various embodiments, lead or lead system 1108 can include a lead similar to lead 1008A but configured to be connected to implantable stimulator 1104 and implantable stimulation units 1105A-N. Implantable stimulator 1104 can represent an example of implantable stimulator 1004 with additional capability of communicating with implantable stimulation units 1105A-N for coordinated pulse delivery. In another embodiment, instead of being separate implantable devices, implantable stimulation units 1105A-N can be incorporated into implantable stimulator 1104 (e.g., implantable stimulator 704 configure to include system 960, with multiple stimulation delivery units each including stimulation output circuit 912). Implantable stimulation units 1105A-N can each generate and deliver neurostimulation pulses in fast succession, for example at VHF, in response to one or more neurostimulation pulses generated from implantable stimulator 1004 (e.g., for every pulse delivered from implantable stimulator 1104, implantable stimulation unit 1105-A can generate and deliver 100 pulses in fast succession). In various embodiments, implantable stimulation units 1105A-N can each receive power from implantable stimulator 1104 or have its own power source.

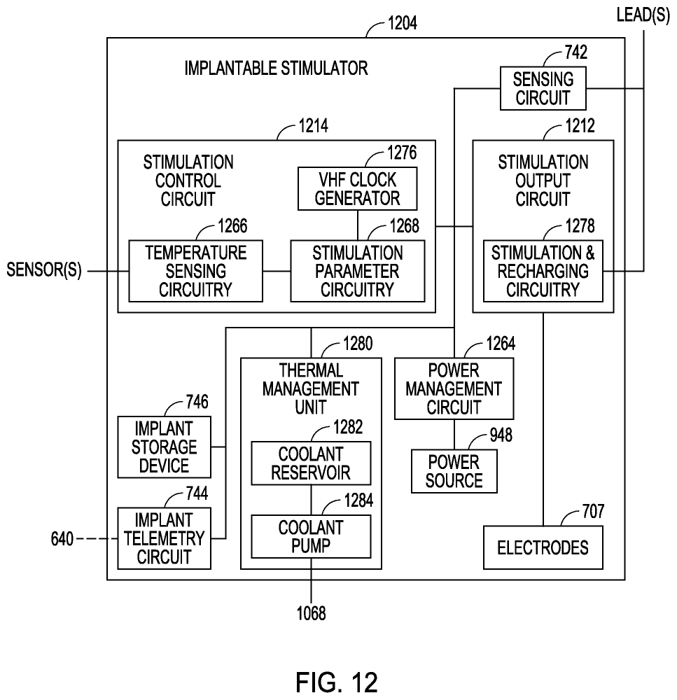

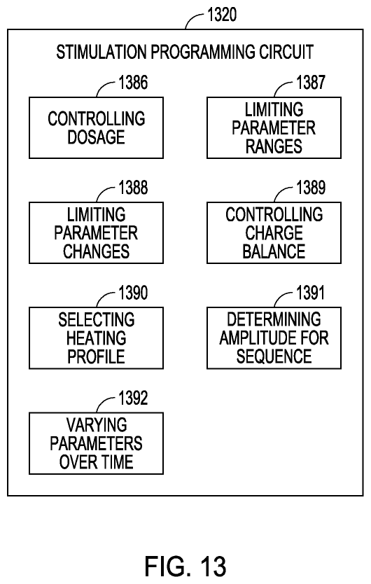

FIG. 12 illustrates an embodiment of an implantable stimulator 1204, which can represent an example of implantable stimulator 704 that includes system 960 (e.g., with specialized hardware and/or firmware to support operation of system 960). Implantable stimulator 1204 can include sensing circuit 742, implant telemetry circuit 744, implant storage device 746, electrode(s) 707, power source 948, a thermal management unit 1280, a stimulation output circuit 1212, a power management circuit 1264, and a stimulation control circuit 1214. In various embodiments, sensor(s) such as sensor(s) 962, each of which may include a sensor circuit, can be incorporated into lead(s) connected to implantable stimulator 1204 and/or implantable stimulator 1204. In various embodiment, heat generated by the neurostimulation delivered from implantable stimulator 1204 through the lead(s) is dissipated over an increased surface area or volume that include not only stimulating electrode and immediately surrounding tissue but also larger areas or volumes accessible through the body of implantable stimulator 1204 and/or one or more implantable heat dissipators such as heat dissipator 1070.

Thermal management unit 1280 can be coupled to a thermal conductive mechanical of a cooling lead, such as thermal conductive conduit 1068 of cooling lead 1008, to control transfer of heat through the cooling lead, for example using the temperature parameter determined by temperature sensing circuitry 966. In the illustrated embodiment, in which thermal conductive conduit 1068 includes a coolant circulation loop, thermal management unit 1280 includes a coolant reservoir 1282 that can store a coolant and a coolant pump 1284 that can cause the coolant to circulate through the coolant circulation loop, thereby transferring the heat through the cooling lead to the housing of implantable stimulator 1204 and/or one or more implantable hear dissipators such that heat dissipator 1070. In other embodiments, a reservoir and/or a pump is not used. In some embodiments, the patient's body motion and/or thermal gradient are used to cause movement of the coolant. Thermal management unit 1280 can control the circulation of the coolant in the coolant circulation loop using the temperature parameter.

Stimulation output circuit 1212 represents an example of stimulation output circuit 912 and can deliver the VHF neurostimulation to the patient through the lead(s). Stimulation output circuit 1212 includes stimulation and recharging circuitry 1278 capable of generating the electrical pulses according to the VHF stimulation parameters and providing for charge balance. In various embodiments, stimulation output circuit 1212 can deliver the VHF neurostimulation according to the pattern of neurostimulation pulses as discussed above with reference to stimulation programming circuit 820, including the various recharge schemes.

Power management circuit 1264 represents an example of power management circuit 964 can cab provide stimulation output circuit 1212 with a power supply signal having a voltage Vh. Power management circuit 1264 can provide suitable Vh levels and related parameters, when necessary, to ensure sufficient and efficient power supply for delivering of the VHF neurostimulation according to the pattern of neurostimulation pulses, which can include various tonic and/or patterned pulses and/or other waveforms. In various embodiments, the load impedance applied to stimulation output circuit 1212 (including lead, tissue, and electrode-tissue interface impedances) may change substantially with the stimulation rate, i.e., the load impedance at VHF may be very different from the load impedance at low stimulation rates. Power management circuit 1264 is designed to maintain Vh level(s) when such substantial changes in the load impedance while delivering the VHF neurostimulation.

Stimulation control circuit 1214 represents an example of stimulation control circuit 914 and can control the delivery of the neurostimulation pulses from stimulation output circuit 1212 using stimulation parameters, such as the stimulation parameters defining the pattern of neurostimulation pulses. Stimulation control circuit 1214 can include a VHF clock generator 1276, temperature sensing circuitry 1266 and stimulation parameter circuitry 1256. VHF clock generator 1276 can generate a high-frequency clock signal for controlling the delivery of VHF neurostimulation with a required or desirable resolution for the stimulation waveform. For example, a clock signal 200 kHz or above may be required for controlling the delivery of neurostimulation pulses at 100 kHz or above. Temperature sensing circuitry 1266 represents an example of temperature sensing circuitry 966 and can be coupled to the sensor(s), such as sensor(s) 962, to receive one or more signals indicative of temperature or temperature change from the sensors(s) and determine a temperature parameter representing the temperature or temperature change using the received one or more signals. Stimulation parameter circuitry 1268 represents an example of stimulation parameter circuitry 968 and can control the delivery of neurostimulation pulses using the temperature parameter. In various embodiments, the temperature parameter can be used as an input for adjusting the stimulation parameters according to a stimulation management routine and/or a closed-loop algorithm. In various embodiments, stimulation parameter circuitry 1268 can control the stimulation parameters for preventing any one or any combination of: (a) the temperature from exceeding a specified threshold temperature (e.g., 40° C.), (b) the temperature from increasing by a specified amount (e.g., 4° C.), and (c) the temperature from staying above a specified threshold temperature, or above an increased amount, for more than a specified duration. The threshold temperature or temperature increase can be increased gradually (e.g., incrementally over several days, as the body develops tolerance to tissue heating). In various embodiments, stimulation parameter circuitry 1268 can adjust the stimulation parameters to limit a dosage based on operational capability of stimulation output circuit 1212 and power management circuit 1264 and a safety limit related to tissue heating. The dosage can be an amount of an electrical energy and/or an amount of electrical charge injected in to the tissue by the delivery of the neurostimulation over a specified duration.

FIG. 13 illustrates an embodiment of a stimulation programming circuit 1320 of a programming device for an implantable system for VHF neurostimulation, such as the implantable system illustrated in FIG. 10. Stimulation programming circuit 1320 represents an example of stimulation programming circuit 820 that is configure to program system 960, such as implemented in implantable stimulator 704, 1004, 1104, or 1204. In various embodiments. stimulation programming circuit 1320 can be part of a user interface including a presentation device and a user input device (such as user interface 810 including presentation device 856 and user input device 858) used as a composer for the pattern of neurostimulation pulses. In the illustrated embodiment, shown by way of example for illustrative but not restrictive purposes, stimulation programming circuit 1320 includes circuitry or functional block for each of controlling dosage 1386, limiting parameter ranges 1387, limiting parameter changes 1388, controlling charge balance 1389, selecting heating profile 1390 determining amplitude for sequence 1391, and varying parameters over time 1392. In various embodiment, can include any one or any combination of the circuitry or functional blocks shown in FIG. 13 and other circuitry or function blocks needed for composing the pattern of neurostimulation pulses for the VHF neurostimulation.

The circuitry or functional block for controlling dosage 1386 can be configured to control dosage (amount of energy or charge delivered over a specified time interval) when composing the pattern of neurostimulation pulses. Various examples of such dosage control can include:
enforcing limits set for the stimulation parameters (e.g., a minimum dosage for ensuring therapeutic efficacy and a maximum dosage for patient safety and/or proper operation of the implantable stimulator);
enforcing a trailing passive recovery delay after a certain amount of energy or charge is delivered (e.g., a non-stimulation period of a specified duration following each stimulation period of a specified duration)|
controlling tissue heating by setting various stimulation parameters to be a function of the temperature parameters to limit an amount of tissue heating (e.g., as measured by increase of tissue temperature) or maintain a measure of tissue heating (e.g., temperature or temperature change) within a desired window (e.g., a range of temperature or a range of temperature change) to keep the measure of tissue heating above a lower limit (minimum) to ensure therapeutic effectiveness but under an upper limit (maximum) to ensure safety;
limiting pulse amplitude to avoid excessive heating (e.g., setting a maximum pulse amplitude for stimulation rate above a specified level or setting the maximum pulse amplitude as a function of the stimulation rate;
limiting the charge injected (i.e., pulse amplitude and pulse width) by each pulse to avoid excessive heating (e.g., setting a maximum charge for stimulation rate above a specified level or setting the maximum pulse charge as a function of the stimulation rate;
determining the stimulation parameters using a map relating the stimulation parameters to tissue heating (e.g., establishing the map relating the dosage to the tissue heating, or establishing the map relating temporal variation of the stimulating parameters, such as an amplitude change profile, to the tissue heating;
managing interactions between the dosage and other timing limitations for the stimulation parameters;
limiting the dosage based on hardware limitations (e.g., to ensure that power management circuit 964 or 1264 can maintain the required level for Vh by producing an alert or warning if the energy requirement of the pattern of neurostimulation pulses exceeds the capability of the implantable stimulator or optimizing the pattern of neurostimulation pulses for maintaining the required Vh, such as by avoiding periods of highly concentrated energy or charge);

The circuitry or functional block for limiting parameter ranges 1387 can limit ranges of one or more stimulation parameters each as a function of the stimulation rate (interlocks). For example, the pulse width is necessarily limited by the stimulation rate. For pulses delivered at 100 kHz, the pulse width is less than 10 us without immediate recharge and shorter if a recharge phase is applied before the next stimulation pulse.

The circuitry or functional block for limiting parameter changes 1388 can limit the speed or frequency of parameter change based on system capability. For example, system 960 may not be capable of accommodating pulse-by-pulse parameter changes at VHF.

The circuitry or functional block for controlling charge balance 1389 can control charge balance. Controlling the charge balance can include limiting the number of pulses and/or the duration with stimulation phases only before recharging and determining a form of recharge (e.g., by select from a list of available recharge schemes, such as those discussed above with reference to stimulation programming circuit 820).

The circuitry or functional block for selecting heating profile 1390 can select a heating profile from a list of heating profiles each associated with a pattern of neurostimulation pulses based on their performance in thermal management. Various patterns of neurostimulation pulse can include examples discussed above (e.g., including stimulation periods for the VHF neurostimulation with non-stimulation periods, with cooling periods, interleaved VHF stimulation, and/or multiple stimulation waveforms). The pattern of neurostimulation pulses may be selected for its heating profile indicating the least amount of temperature increase.

The circuitry or functional block for determining amplitude for sequence 1391 can determine amplitude for a sequence of neurostimulation pulses (e.g., an entire pattern of neurostimulation pulses). A calibration can be tuned to a specified effect (e.g., verifying for a specified number of times that heating profile changes with pulse amplitude as expected) for determining an overall pulse amplitude for the sequence.

The circuitry or functional block for varying parameters over time 1392 can vary stimulation parameters over time to avoid undesired effects. Various stimulation parameters can be dynamically adjusted to vary the stimulation field (e.g., selection of electrodes and/or fractionalization), the stimulation waveform (e.g., pulse amplitude, pulse width, pulse rate), and/or durations of non-stimulation periods. For example, the stimulation filed can be set to vary over time to prevent excessive local tissue heating. This can be done by using stimulation field models (SFMs) relating stimulation fields to volumes of activation (VOAs) to determine multiple stimulation fields suitable for delivering the VHF neurostimulation. In some embodiments, particular or differentiated SFMs are made for VHF neurostimulation. In some examples, the SFM can be scaled according the stimulation frequency, and in other examples, different SFMs can be used for different frequencies, frequency ranges, and/or frequency combinations. In some examples, the stimulation frequency used for selecting or adjusting the SFMs can be a function of the frequency deliverable by the stimulation device, and can depend on the properties of the stimulation waveforms used. In various embodiments, specialized circuitry and/or algorithm can be configured to ensure delivery of the VHF neurostimulation without introducing artifact and/or switching noise. Such artifact and/or switching noise may result in delivery of stimulation with unintended waveform shapes to the tissue, such as spikes on leading and trailing edges of pulses and/or ripples in the steady-state values during an active pulse.

In various embodiments, a non-transitory computer-readable storage medium includes instructions, which when executed by a system (e.g., system 960), cause the system to perform any one or any combination of the functions of stimulation programming circuit 1320 including circuitry or functional block for each of controlling dosage 1386, limiting parameter ranges 1387, limiting parameter changes 1388, controlling charge balance 1389, selecting heating profile 1390 determining amplitude for sequence 1391, and varying parameters over time 1392. Examples of such storage medium include external storage device 818 and/or any storage medium used for configuring (e.g., programming) an implantable stimulator (e.g., implantable stimulator 1204) and/or external programming device (e.g., external programming device 802).

In addition to those discussed in the SUMMARY section above, nonlimiting examples of the present subject matter are provided as follows:

### (System for VHF Neurostimulation)

In Example 1, a system for delivering neurostimulation to one or more regions of tissue in a patient is provided. The system may include a stimulation output circuit, a power management circuit, and a stimulation control circuit. The stimulation output circuit may be configured to deliver the neurostimulation using a very-high-frequency (VHF) stimulation waveform to the patient. The VHF stimulation waveform may have a frequency of, for example, at least 100 kHz. The power management circuit may be configured to provide the stimulation output circuit with a power supply signal and to maintain a specified voltage level for the power supply signal. The stimulation control circuit may be configured to control the delivery of the neurostimulation using stimulation parameters.

In Example 2, the subject matter of Example 1 may optionally be configured to further include a stimulation programming circuit configured to determine a pattern of neurostimulation pulses defined by the stimulation parameters, and may optionally be configured such that the stimulation control circuit is configured to control the delivery of the neurostimulation according to the pattern of neurostimulation pulses.

In Example 2, the subject matter of Example 2 may optionally be configured to include an implantable neurostimulator including at least the stimulation output circuit, the power management circuit, and the stimulation control circuit.

In Example 4, the subject matter of Example 3 may optionally be configured to include an external programmer configured to be communicatively coupled to the implantable neurostimulator. The external programmer includes the stimulation programming circuit.

### (Thermal Management System)

In Example 5, the subject matter of any one or any combination of Examples 3 and 4 may optionally be configured to further include one or more sensors configured to be placed in the patient to sense a measure of tissue heating caused by the delivery of the neurostimulation, and may optionally be configured such that such that the stimulation control circuit includes temperature sensing circuitry and stimulation parameter circuitry. The temperature sensing circuitry is configured to receive the measure of tissue heating and to determine a temperature parameter representing a temperature or a temperature change using the received measure of tissue heating. The stimulation parameter circuitry is configured to adjust the stimulation parameters using the temperature parameter and to adjust the stimulation parameters to limit a dosage based on an operational capability of the stimulation output circuit and the power management circuit and a safety limit related to tissue heating, the dosage including an amount of at least one of an electrical energy or electrical charge injected in to the tissue by the delivery of the neurostimulation over a specified duration.

In Example 6, the subject matter of Example 5 may optionally be configured to further include one or more heat dissipators, a thermal management device configured to be coupled to the one or more heat dissipators, and a thermal conductive lead having a proximal end configured to be coupled to the thermal management device and a distal end configured to be placed in or about a region of the one or more regions of tissue, and may optionally be configured such that the thermal management device is configured to provide a thermal conductive path for a portion of a thermal energy causing the tissue heating to be dissipated through at least the one or more heat dissipators.

In Example 7, the subject matter of Example 6 may optionally be configured such that the implantable neurostimulator further includes the thermal management device.

In Example 8, the subject matter of Example 7 may optionally be configured such that the one or more heat dissipators includes at least one implantable heat dissipator configured to be coupled to the implantable neurostimulator.

In Example 9, the subject matter of any one or any combination of Examples 7 and 8 may optionally be configured such that the implantable neurostimulator has a case housing at least the stimulation output circuit, the power management circuit, the stimulation control circuit, and the thermal management device, and may optionally be configured such that the one or more heat dissipators include at least the case.

In Example 10, the subject matter of any one or any combination of Examples 6 to 9 may optionally be configured to further include one or more implantable leads each including a proximal end configured to be coupled to the implantable neurostimulator, a distal end configured to be placed in or about a region of the one or more regions of tissue, and a plurality of electrodes at the distal end, and may optionally be configured such that at least one of the one or more implantable leads is configured to be the thermal conductive lead, the one or more sensors are each incorporated into the distal end of a lead of the one or more implantable leads, and the stimulation output circuit is configured to deliver the neurostimulation to the one or more regions of tissue using one or more electrodes selected from the plurality of electrodes at the distal end of each lead of the one or more implantable leads.

In Example 11, the subject matter of any one or any combination of Examples 6 to 10 may optionally be configured to further include a coolant circulation path formed in the one or more heat dissipators, the thermal management device and the thermal conductive lead, and may optionally be configured such that the thermal management device includes a coolant reservoir coupled to the coolant circulation path and configured to store a coolant and a coolant pump coupled to the coolant circulation path and configured to control movement of the coolant in the coolant circulation path.

In Example 12, the subject matter of any one or any combination of Examples 5 to 11 may optionally be configured such that the one or more sensors includes at least one temperature sensor configured to sense a temperature directly.

In Example 13, the subject matter of any one or any combination of Examples 5 to 12 may optionally be configured such that the one or more sensors includes at least one physiological sensor configured to sense a physiological signal indicative of a temperature or a change of the temperature.

### (Stimulation Device)

In Example 14, the subject matter of any one or any combination of Examples 3 to 13 may optionally be configured such that the implantable neurostimulator includes multiple implantable devices communicatively coupled to each other to increase a maximum frequency for the VHF stimulation waveform.

In Example 15, the subject matter of any one or any combination of Examples 1 to 14 may optionally be configured such that the stimulation output circuit is configured to allow for delivering neurostimulation pulses with stimulation and recharge phases for charge balancing using a recharge scheme selectable from a plurality of recharge schemes.

In Example 16, the subject matter of any one or any combination of Examples 5 to 15 may optionally be configured such that the stimulation parameter circuitry is configured to adjust the stimulation parameters using the temperature parameter to prevent the temperature or the temperature change from exceeding a specified threshold.

In Example 17, the subject matter of any one or any combination of Examples 5 to 16 may optionally be configured such that the stimulation parameter circuitry is configured to adjust the stimulation parameters using the temperature parameter to maintain the temperature or the temperature change within a specified range.

### (Programming Device for Determining Pattern of Neurostimulation Pulses)

In Example 18, the subject matter of any one or any combination of Examples 2 to 17 may optionally be configured such that the stimulation programming circuit is configured to compose the pattern of neurostimulation pulses to include one or more stimulation periods during which one or more pulses of the pattern of neurostimulation pulses are delivered and one or more non-stimulation periods during which no pulse of the pattern of neurostimulation pulses is delivered. At least one stimulation period of the one or more stimulation periods includes bursts of neurostimulation pulses at a VHF pulse rate.

In Example 19, the subject matter of Example 18 may optionally be configured such that the stimulation programming circuit is configured to compose the pattern of neurostimulation pulses to include one or more cooling periods each being a non-stimulation period of the one or more non-stimulation periods or a stimulation period of the one or more stimulation periods during which the neurostimulation is delivered to a region of the one or more regions of tissue for cooling a different region of the one or more regions of tissue.

In Example 20, the subject matter of any one or any combination of Examples 2 to 19 may optionally be configured such that the stimulation programming circuit is configured to compose the pattern of neurostimulation pulses to include neurostimulation pulses at a VHF pulse rate of greater than about 100 kHz interleaved with neurostimulation pulses at low pulse rate of lower than about 1.2 kHz.

In Example 21, the subject matter of any one or any combination of Examples 2 to 20 may optionally be configured such that the stimulation programming circuit is configured to compose the pattern of neurostimulation pulses to include multiple types of stimulation waveforms.

In Example 22, the subject matter of any one or any combination of Examples 2 to 21 may optionally be configured such that the stimulation programming circuit is configured to compose the pattern of neurostimulation pulses to include recharge periods each balancing electrical charges of one or more pulses of the pattern of neurostimulation pulses by using one or more recharge pulses each being an active recharge pulse or a passive recharge pulse.

(Programming Device with Pattern Composition Rules)

In Example 23, the subject matter of any one or any combination of Examples 5 to 22 may optionally be configured such that the stimulation programming circuit is configured to determine the pattern of neurostimulation pulses to allow the stimulation parameter circuitry of the stimulation control circuit to limit the dosage while providing a minimum dosage for ensuring therapeutic efficacy of the neurostimulation.

In Example 24, the subject matter of Example 23 may optionally be configured such that the stimulation programming circuit is configured to determine the pattern of neurostimulation pulses using a map relating the stimulation parameters defining the pattern of neurostimulation pulses to tissue heating.

In Example 25, the subject matter of Example 24 may optionally be configured such that the stimulation programming circuit is configured to determine the pattern of neurostimulation pulses using one or more parameter profiles each relates temporal variation of a parameter of the stimulation parameters defining the pattern of neurostimulation pulses to the tissue heating.

In Example 26, the subject matter of any one or any combination of Examples 23 to 25 may optionally be configured such that the stimulation programming circuit is configured to determine the pattern of neurostimulation pulses to allow the stimulation parameter circuitry of the stimulation control circuit to limit the dosage based on a limit of power available to the power management circuit.

In Example 27, the subject matter of any one or any combination of Examples 23 to 26 may optionally be configured such that the stimulation programming circuit is configured to limit a range for each stimulation parameter of the stimulation parameters defining the pattern of neurostimulation pulses as a function of a maximum pulse rate in the pattern of neurostimulation pulses.

In Example 28, the subject matter of any one or any combination of Examples 23 to 27 may optionally be configured such that the stimulation programming circuit is configured to limit at least one of a speed, a frequency, or a magnitude of value change of a parameter of the stimulation parameters defining the pattern of neurostimulation pulses based on the operational capability of the stimulation output circuit and the power management circuit.

In Example 29, the subject matter of any one or any combination of Examples 23 to 25 may optionally be configured such that the stimulation programming circuit is configured to control charge balance of the pattern of neurostimulation pulses, including limiting a number of pulses delivered without introducing one or more recharge pulses.

In Example 30, the subject matter of any one or any combination of Examples 23 to 29 may optionally be configured such that the stimulation programming circuit is configured to limit the dosage by temporally distributing the dosage using varying stimulation waveforms defined by the stimulation parameters defining the pattern of neurostimulation pulses.

In Example 31, the subject matter of any one or any combination of Examples 23 to 30 may optionally be configured such that the stimulation programming circuit is configured to limit the dosage by spatially distributing the dosage using varying stimulation fields defined by the stimulation parameters defining the pattern of neurostimulation pulses.

### (Method for VHF Neurostimulation)

In Example 32, a method for delivering neurostimulation to one or more regions of tissue in a patient is provided. The method may include delivering the neurostimulation from a stimulation output circuit using a very-high-frequency (VHF) stimulation waveform to the patient, providing the stimulation output circuit with a power supply signal, including maintaining a specified voltage level for the power supply signal, and controlling the delivery of the neurostimulation using stimulation parameters. The VHF stimulation waveform has a frequency of, for example, at least 100 kHz;

In Example 33, the subject matter of Examples 32 may optionally further include determining a pattern of neurostimulation pulses defined by the stimulation parameters, and the subject matter of controlling the delivery of the neurostimulation using the stimulation parameters as found in Example 32 may optionally include controlling the delivery of the neurostimulation according to the pattern of neurostimulation pulses.

In Example 34, the subject matter of delivering the neurostimulation from the stimulation output circuit as found in Example 33 may optionally include delivering the neurostimulation from the stimulation output circuit of an implantable neurostimulator.

In Example 35, the subject matter of determining the pattern of neurostimulation pulses as found in Example 34 may optionally include determining the pattern of neurostimulation pulses using an external programmer communicatively coupled to the implantable neurostimulator.

### (Thermal Management Method)

In Example 36, the subject matter of any one or any combination of Examples 34 and 35 may optionally further include sensing a measure of tissue heating caused by the delivery of the neurostimulation using one or more sensors, determining a temperature parameter representing a temperature or a temperature change using the received measure of tissue heating, adjusting the stimulation parameters using the temperature parameter, and adjusting the stimulation parameters to limit a dosage based on an operational capability of the implantable neurostimulator and a safety limit related to tissue heating. The dosage includes an amount of at least one of an electrical energy or electrical charge injected in to the tissue by the delivery of the neurostimulation over a specified duration.

In Example 37, the subject matter of Example 36 may optionally further include providing one or more heat dissipators, providing the implantable neurostimulator with a thermal management device configured to be coupled to the one or more heat dissipators, providing a thermal conductive lead having a proximal end configured to be coupled to the thermal management device and a distal end configured to be placed in or about a region of the one or more regions of tissue, and providing a thermal conductive path using the thermal management device and the thermal conductive lead for a portion of a thermal energy causing the tissue heating to be dissipated through at least the one or more heat dissipators.

In Example 38, the subject matter of providing the one or more heat dissipators as found in Example 37 may optionally include providing at least one implantable heat dissipator configured to be coupled to the implantable neurostimulator.

In Example 39, the subject matter of providing the one or more heat dissipators as found in any one or any combination of Examples 34 and 35 may optionally include using a portion of the implantable neurostimulator as a heat dissipator of the one or more heat dissipators.

In Example 40, the subject matter of any one or any combination of Examples 37 to 39 may optionally further include connecting one or more implantable leads to the implantable neurostimulator. The one or more implantable leads each include a proximal end configured to be coupled, a distal end configured to be placed in or about a region of the one or more regions of tissue, and a plurality of electrodes at the distal end. At least one of the one or more implantable leads is configured to be the thermal conductive lead. The one or more sensors are each incorporated into the distal end of a lead of the one or more implantable leads. The subject matter of delivering the neurostimulation as found in any one or any combination of Examples 37 to 39 may optionally include delivering the neurostimulation using one or more electrodes selected from the plurality of electrodes at the distal end of each lead of the one or more implantable leads.

In Example 41, the subject matter of any one or any combination of Examples 37 to 40 may optionally further include forming a coolant circulation path in the one or more heat dissipators, the thermal management device, and the thermal conductive lead, and the subject matter of providing the implantable stimulator with the thermal management device as found in any one or any combination of Examples 37 to 40 may optionally include providing a thermal management device including a coolant reservoir coupled to the coolant circulation path and configured to store a coolant and a coolant pump coupled to the coolant circulation path and configured to control movement of the coolant in the coolant circulation path.

In Example 42, the subject matter of sensing the measure of tissue heating caused by the delivery of the neurostimulation as found in any one or any combination of Examples 35 to 41 may optionally include sensing a temperature directly.

In Example 43, the subject matter of sensing the measure of tissue heating caused by the delivery of the neurostimulation as found in any one or any combination of Examples 35 to 42 may optionally include sensing a physiological signal indicative of a temperature or a change of the temperature.

### (Method for Operating Stimulation Device)

In Example 44, the subject matter of delivering the neurostimulation as found in any one or any combination of Examples 34 to 43 may optionally include delivering the neurostimulation from multiple implantable devices communicatively coupled to each other to increase a maximum frequency for the VHF stimulation waveform.

In Example 45, the subject matter of delivering the neurostimulation as found in any one or any combination of Examples 34 to 44 may optionally include delivering neurostimulation pulses with stimulation and recharge phases for charge balancing using a recharge scheme selectable from a plurality of recharge schemes.

In Example 46, the subject matter of any one or any combination of Examples 34 to 45 may optionally further include adjusting the stimulation parameters using the temperature parameter to prevent the temperature or the temperature change from exceeding a specified threshold.

In Example 47, the subject matter of any one or any combination of Examples 34 to 46 may optionally further include adjusting the stimulation parameters using the temperature parameter to maintain the temperature or the temperature change within a specified range.

### (Method for Determining Pattern of Neurostimulation Pulses)

In Example 48, the subject matter of any one or any combination of Examples 34 to 47 may optionally further include composing the pattern of neurostimulation pulses to include one or more stimulation periods during which one or more pulses of the pattern of neurostimulation pulses are delivered and one or more non-stimulation periods during which no pulse of the pattern of neurostimulation pulses is delivered, at least one stimulation period of the one or more stimulation periods including bursts of neurostimulation pulses at a VHF pulse rate.

In Example 49, the subject matter of Example 48 may optionally further include composing the pattern of neurostimulation pulses to include one or more cooling periods each being a non-stimulation period of the one or more non-stimulation periods or a stimulation period of the one or more stimulation periods during which the neurostimulation is delivered to a region of the one or more regions of tissue for cooling a different region of the one or more regions of tissue.

In Example 50, the subject matter of any one or any combination of Examples 33 to 49 may optionally further include composing the pattern of neurostimulation pulses to include neurostimulation pulses at a VHF pulse rate of greater than about 100 kHz interleaved with neurostimulation pulses at low pulse rate of lower than about 1.2 kHz.

In Example 51, the subject matter of any one or any combination of Examples 33 to 50 may optionally further include composing the pattern of neurostimulation pulses to include multiple types of stimulation waveforms.

In Example 52, the subject matter of any one or any combination of Examples 33 to 51 may optionally further include composing the pattern of neurostimulation pulses to include recharge periods each balancing electrical charges of one or more pulses of the pattern of neurostimulation pulses by using one or more recharge pulses each being an active recharge pulse or a passive recharge pulse.

(Method for Complying with Pattern Composition Rules)

In Example 53, the subject matter of any one or any combination of Examples 36 to 52 may optionally further include determining the pattern of neurostimulation pulses to limit the dosage while providing a minimum dosage for ensuring therapeutic efficacy of the neurostimulation.

In Example 54, the subject matter of determining the pattern of neurostimulation pulses as found in Example 53 may optionally include determining the pattern of neurostimulation pulses using a map relating the stimulation parameters defining the pattern of neurostimulation pulses to tissue heating.

In Example 55, the subject matter of determining the pattern of neurostimulation pulses as found in Example 54 may optionally include determining the pattern of neurostimulation pulses using one or more parameter profiles each relates temporal variation of a parameter of the stimulation parameters defining the pattern of neurostimulation pulses to the tissue heating.

In Example 56, the subject matter of determining the pattern of neurostimulation pulses as found in any one or any combination of Examples 53 to 55 may optionally include determining the pattern of neurostimulation pulses to allow the dosage to be limited based on a limit of power available to the stimulation output circuit.

In Example 57, the subject matter of determining the pattern of neurostimulation pulses as found in any one or any combination of Examples 53 to 56 may optionally include limiting a range for each stimulation parameter of the stimulation parameters defining the pattern of neurostimulation pulses as a function of a maximum pulse rate in the pattern of neurostimulation pulses.

In Example 58, the subject matter of determining the pattern of neurostimulation pulses as found in any one or any combination of Examples 53 to 57 may optionally include limiting at least one of a speed, a frequency, or a magnitude of value change of a parameter of the stimulation parameters defining the pattern of neurostimulation pulses based on the operational capability of the stimulation output circuit and the power management circuit.

In Example 59, the subject matter of determining the pattern of neurostimulation pulses as found in any one or any combination of Examples 53 to 58 may optionally include controlling charge balance of the pattern of neurostimulation pulses, including limiting a number of pulses delivered without introducing one or more recharge pulses.

In Example 60, the subject matter of determining the pattern of neurostimulation pulses as found in any one or any combination of Examples 53 to 59 may optionally include limiting the dosage by temporally distributing the dosage using varying stimulation waveforms defined by the stimulation parameters defining the pattern of neurostimulation pulses.

In Example 61, the subject matter of determining the pattern of neurostimulation pulses as found in any one or any combination of Examples 53 to 60 may optionally include limiting the dosage by spatially distributing the dosage using varying stimulation fields defined by the stimulation parameters defining the pattern of neurostimulation pulses.

### (Storage Medium for Instructions to Perform VHF Neurostimulation)

In Example 62, a non-transitory computer-readable storage medium including instructions, which when executed by a system, cause the system to perform a method delivering neurostimulation from a stimulation device to a patient is provided. The neurostimulation is delivered from a stimulation output circuit of the stimulation device using a very-high-frequency (VHF) stimulation waveform. The VHF stimulation waveform has a frequency of, for example, at least 100 kHz. The method may include providing the stimulation output circuit with a power supply signal and maintaining a specified voltage level for the power supply signal during the delivery of the neurostimulation.

In Example 63, the method as found in Example 61 may optionally further include the subject matter of any one or any combination of Examples 33-36 and 45-61.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering neurostimulation to one or more regions of tissue in a patient, the system comprising:
one or more sensors configured to be placed in the patient to sense a measure of tissue heating caused by the delivery of the neurostimulation;
a stimulation output circuit configured to deliver the neurostimulation; and
a stimulation control circuit configured to control the delivery of the neurostimulation using stimulation parameters, the stimulation control circuit including:
temperature sensing circuitry configured to receive the measure of tissue heating and to determine a temperature parameter representing a temperature or a temperature change using the received measure of tissue heating; and
stimulation parameter circuitry configured to adjust the stimulation parameters using the temperature parameter and to adjust the stimulation parameters to limit a dosage based on an operational capability of the stimulation output circuit and the power management circuit and a safety limit related to tissue heating, the dosage including an amount of at least one of an electrical energy or electrical charge injected in to the tissue by the delivery of the neurostimulation over a specified duration.

2. The system of claim 1, wherein the stimulation output circuit is configured to deliver the neurostimulation using a very-high-frequency (VHF) stimulation waveform to the patient, the VHF stimulation waveform having a frequency of at least 100 KHz.

3. The system of claim 2, comprising an implantable neurostimulator including at least the stimulation output circuit, the power management circuit, and the stimulation control circuit, and further comprising:
one or more heat dissipators;
a thermal management device configured to be coupled to the one or more heat dissipators; and
a thermal conductive lead having a proximal end configured to be coupled to the thermal management device and a distal end configured to be placed in or about a region of the one or more regions of tissue,
wherein the thermal management device is configured to provide a thermal conductive path for a portion of a thermal energy causing the tissue heating to be dissipated through at least the one or more heat dissipators.

4. The system of claim 3, wherein the implantable neurostimulator further comprises the thermal management device and has a case housing at least the stimulation output circuit, the power management circuit, the stimulation control circuit, and the thermal management device, and the one or more heat dissipators comprise at least one of an implantable heat dissipator configured to be coupled to the implantable neurostimulator or a portion of the case.

5. The system of claim 3, further comprising one or more implantable leads each including a proximal end configured to be coupled to the implantable neurostimulator, a distal end configured to be placed in or about a region of the one or more regions of tissue, and a plurality of electrodes at the distal end, wherein:
at least one of the one or more implantable leads is configured to be the thermal conductive lead;
the one or more sensors are each incorporated into the distal end of a lead of the one or more implantable leads; and
the stimulation output circuit is configured to deliver the neurostimulation to the one or more regions of tissue using one or more electrodes selected from the plurality of electrodes at the distal end of each lead of the one or more implantable leads.

6. The system of claim 3, further comprising a coolant circulation path formed in the one or more heat dissipators, the thermal management device, and the thermal conductive lead, and wherein the thermal management device comprises:
a coolant reservoir coupled to the coolant circulation path and configured to store a coolant; and
a coolant pump coupled to the coolant circulation path and configured to control movement of the coolant in the coolant circulation path.

7. The system of claim 3, wherein the implantable neurostimulator comprises multiple implantable devices communicatively coupled to each other to increase a maximum frequency for the VHF stimulation waveform.

8. The system of claim 1, wherein the one or more sensors comprise at least one physiological sensor configured to sense a physiological signal indicative of a temperature or a change of the temperature.

9. The system of claim 1, wherein the stimulation parameter circuitry is configured to adjust the stimulation parameters using the temperature parameter to prevent the temperature or the temperature change from exceeding a specified threshold.

10. The system of claim 1, wherein the stimulation parameter circuitry is configured to adjust the stimulation parameters using the temperature parameter to maintain the temperature or the temperature change within a specified range.

11. A method for delivering neurostimulation to one or more regions of tissue in a patient, the method comprising:
delivering the neurostimulation from a stimulation output circuit from a stimulation device;
sensing a measure of tissue heating caused by the delivery of the neurostimulation using one or more sensors;
determining a temperature parameter representing a temperature or a temperature change using the received measure of tissue heating;
adjusting the stimulation parameters using the temperature parameter; and
adjusting the stimulation parameters to limit a dosage based on an operational capability of the implantable neurostimulator and a safety limit related to tissue heating, the dosage including an amount of at least one of an electrical energy or electrical charge injected in to the tissue by the delivery of the neurostimulation over a specified duration.

12. The method of claim 11, wherein delivering the neurostimulation comprises delivering the neurostimulation using a very-high-frequency (VHF) stimulation waveform, the VHF stimulation waveform having a frequency of at least 100 KHz.

13. The method of claim 12, wherein delivering the neurostimulation comprises delivering the neurostimulation using an implantable neurostimulator.

14. The method of claim 13, further comprising:
providing one or more heat dissipators;
providing the implantable neurostimulator with a thermal management device configured to be coupled to the one or more heat dissipators;
providing a thermal conductive lead having a proximal end configured to be coupled to the thermal management device and a distal end configured to be placed in or about a region of the one or more regions of tissue; and
providing a thermal conductive path using the thermal management device and the thermal conductive lead for a portion of a thermal energy causing the tissue heating to be dissipated through at least the one or more heat dissipators.

15. The method of claim 14, wherein providing the one or more heat dissipators comprises providing at least one implantable heat dissipator configured to be coupled to the implantable neurostimulator.

16. The method of claim 14, wherein providing the one or more heat dissipators comprises using a portion of the implantable neurostimulator as a heat dissipator of the one or more heat dissipators.

17. The method of claim 14, further comprising connecting one or more implantable leads to the implantable neurostimulator, the one or more implantable leads each including a proximal end configured to be coupled, a distal end configured to be placed in or about a region of the one or more regions of tissue, and a plurality of electrodes at the distal end, wherein:
at least one of the one or more implantable leads is configured to be the thermal conductive lead;
the one or more sensors are each incorporated into the distal end of a lead of the one or more implantable leads; and
delivering the neurostimulation comprises delivering the neurostimulation using one or more electrodes selected from the plurality of electrodes at the distal end of each lead of the one or more implantable leads.

18. The method of claim 14, further comprising forming a coolant circulation path in the one or more heat dissipators, the thermal management device, and the thermal conductive lead, and wherein providing the implantable stimulator with the thermal management device comprises providing a thermal management device including:
a coolant reservoir coupled to the coolant circulation path and configured to store a coolant; and
a coolant pump coupled to the coolant circulation path and configured to control movement of the coolant in the coolant circulation path.

19. The method of claim 13, wherein delivering the neurostimulation comprises delivering the neurostimulation from multiple implantable devices communicatively coupled to each other to increase a maximum frequency for the VHF stimulation waveform.

20. A non-transitory computer-readable storage medium including instructions, which when executed by a system, cause the system to perform a method for delivering neurostimulation to one or more regions of tissue in a patient, the method comprising:
delivering the neurostimulation from a stimulation output circuit;
receiving a measure of tissue heating caused by the delivery of the neurostimulation, the measure of tissue heating sensed using one or more sensors;
determining a temperature parameter representing a temperature or a temperature change using the received measure of tissue heating;
adjusting the stimulation parameters using the temperature parameter; and
adjusting the stimulation parameters to limit a dosage based on an operational capability of the implantable neurostimulator and a safety limit related to tissue heating, the dosage including an amount of at least one of an electrical energy or electrical charge injected in to the tissue by the delivery of the neurostimulation over a specified duration.
